(19) United States
(12) Patent Application Publication
(10) Pub. No.: US 2023/0273166 A1
(43) Pub. Date: Aug. 31, 2023

(54) SYSTEM FOR ELECTROPHYSIOLOGICAL ANALYSIS OF PLANTS

(71) Applicant: Vegetal Signals, Bordeaux (FR)

(72) Inventors: Patrice Cocheteux, Bordeaux (FR); Fabian Le Bourdiec, Bordeaux (FR)

(21) Appl. No.: 18/016,061

(22) PCT Filed: Jul. 7, 2021
(86) PCT No.: PCT/EP2021/068889

§ 371 Date: Jan. 13, 2023

(30) Foreign Application Priority Data
Jul. 15, 2020 (FR) FR2007385
Jul. 15, 2020 (FR) FR2007386
Jul. 15, 2020 (FR) FR2007387

(51) Int. Cl. G01N 33/00

(52) U.S. Cl. CPC G01N 33/0098; A01G 7/04

(57) ABSTRACT

The invention relates to a system for electrophysiological analysis of plants, characterized in that it comprises: a plurality of acquisition systems, each acquisition system comprising: —a plurality of pairs of electrodes each comprising a first electrode and a second electrode each intended to be connected to the same plant in order to pick up first and second analog electric potentials, a plurality of operational amplifiers arranged in order to measure the differences in potential between the first and second analog electric potentials acquired by the first and second electrodes of said acquisition system and a plurality of analog-to-digital converters designed to convert the differences in potential into digital signals, —a central data collection system comprising a memory and a source of electrical power, characterized in that each acquisition system is connected to the central system and comprises: a connecting unit designed to transmit the digital signals to the central system, a power unit designed to transmit the electrical power obtained from the electrical energy source to the operational amplifiers and to the analog-to-digital converters, each acquisition system being designed to galvanically isolate the operational amplifiers and the analog-to-digital converters from the power unit and the connecting unit.

The invention relates to the field of the electrophysiological analysis of plants. More specifically, the invention relates to a system for electrophysiological analysis of a plurality of plants.

It has been observed that plants emit electrical signals, in particular in the form of action potentials which express their physiological state. By measuring these electrical signals by means of electrodes and analyzing said signals, it is therefore possible to determine the physiological state of a plant faster and more reliably than by direct means of observation. Moreover, if a situation of stress or disease is detected, it is also possible to propose corrective actions immediately and automatically allowing said situation to be remedied.

However, existing systems for electrophysiological analysis of plants are designed for use in the laboratory and are not suitable for agricultural use. This is because the known systems are based, on the one hand, on a system for acquiring electrical signals that allows those signals to be digitized for processing and on the other hand on a module for processing the digitized signals which uses in particular statistical analysis methods. It is therefore necessary to acquire a large number of electrical signals from a plurality of plants simultaneously and over long periods. Moving to the field scale leads to a plurality of constraints. Firstly, a plurality of measurement points must be deployed, in particular some hundreds of measurement points, over a large area, in particular over some thousands of square meters. It is therefore necessary for all these measurement points to be able to be deployed easily and to have a simple design in order to present a low installation and operational cost.

Secondly, it must be possible to supply the electronics with power allowing the electrical signals obtained at the measurement points to be acquired and digitized. However, this multiplicity of measurement points will consequently require the use of a great many supply cables in order to supply each of the acquisition devices and data transmission cables with power to allow the digitized signals to be transmitted to the processing module. The number of cables thus required has a negative impact on the robustness of the system, its cost and ease of installation and use.

Finally, the electrical signals emitted by plants are of very low intensity, approximately a millivolt. But the electrical supply to the acquisition and digitization electronics is liable to disturb or indeed have a negative impact on said acquisition and digitization, which could distort the measurement and the data.

The present invention is thus positioned in this context and therefore aims to respond to the needs referred to while overcoming the drawbacks mentioned.

Accordingly, the invention relates to a system for electrophysiological analysis of plants, characterized in that it comprises:
a. a plurality of acquisition systems, each acquisition system comprising:
   i. a plurality of pairs of electrodes each comprising a first electrode and a second electrode each intended to be connected to the same plant in order to pick up first and second analog electric potentials;
   ii. a plurality of operational amplifiers arranged in order to measure the differences in potential between the first and second analog electric potentials acquired by the first and second electrodes of said acquisition system and a plurality of analog-to-digital converters designed to convert the differences in potential into digital signals;
b. a central data collection system comprising a memory and a source of electrical power;
and in that each acquisition system is connected to the central system and comprises:
c. a connecting unit designed to transmit the digital signals to the central system;
d. a power unit designed to transmit electrical power obtained from the electrical energy source to the operational amplifiers and to the analog-to-digital converters;
and in which each acquisition system is designed to galvanically isolate the operational amplifiers and the analog-to-digital converters from the power unit and the connecting unit.

According to the invention, a central system is arranged on the one hand to collect the data coming from the acquisition systems and to transmit electric power to each of these acquisition systems. This star architecture, organized around the central system, allows the number of measurement points to be substantially increased while reducing the number of cables and centralizing the electrical energy source. The measurement points and the active electronics of each acquisition system may therefore be designed and organized according to the topology that best suits the field in which said acquisition systems are to be deployed, each acquisition system ensuring that the electric power supplied by the central system is transmitted to the active electronics while avoiding causing interference or influencing the data generated by the active electronics.

Preferably, each pair of electrodes may be connected to a plant so as to measure a linear electric field generated by the superposition of temporary depolarizations of the membranes of the leaf cells and/or by ion exchanges in the region of the root system. For example, each of the first and second electrodes may be connected to the stem of the plant in order to be in contact with the phloem or, in a variant, one of the electrodes may be connected to the stem of the plant and the other electrode may be connected to a subterranean bulb of the plant. If applicable, each electrode is a needle electrode. This type of electrode is particularly suitable for agricultural use, the connection of a needle electrode to the plant being more robust than that of a cupula electrode in a context where the plants are likely to grow and undergo mechanical constraints, in particular caused by wind.

If applicable, each second electrode of an acquisition system is connected to a common reference electric potential, in particular a ground of a portion of the acquisition system, isolated from the power unit and from the connecting unit.

Advantageously, each acquisition system comprises a plurality of terminals each connected to a group of electrode pairs of the acquisition system and arranged to collect said analog electric potentials picked up by the first and second electrodes of that group, and a relay connected to said terminals of the acquisition system and to the central system, said relay comprising said connecting unit and said power unit. If applicable, the operational amplifiers and the analog-to-digital converters of each acquisition system are provided in the relay and/or in the terminals of the acquisition system, and the relay of each acquisition system is arranged to galvanically isolate the operational amplifiers and the analog-to-digital converters of the acquisition system from its power unit and from its connecting unit. In this embodiment, each acquisition system therefore has a tree topology with the root formed by the relay and the branches extending to the electrode pairs. For each acquisition system, provision may be made for the terminals to be connected directly to the relay, the tree in this case having only two levels, or alternatively for the terminals to be connected by extenders in order to reach more distant measurement points, or by concentrators or hubs in order to reduce the number of terminals, in which case the tree may have more than two levels.

Preferably, each second electrode of each acquisition system is connected to a common reference electric potential, in particular a ground of a relay of said acquisition system, isolated from the power unit and from the connecting unit of said relay.

If applicable, each first electrode is associated with a filter arranged to filter the first analog electric potential picked up by said first electrode. The or each filter may comprise an analog smoothing filter and/or a protection filter against electrostatic discharges.

In an embodiment of the invention, each terminal of each acquisition system is arranged to transmit the analog electric potentials that it collects to the relay of the acquisition system. If applicable, the relay of each acquisition system comprises a plurality of acquisition stages each connected to at least one of the terminals of said acquisition system to receive said collected analog electric potentials and comprising at least an operational amplifier and an analog-to-digital converter to measure said differences in potential and convert said differences in potential into digital signals, each acquisition stage being connected to the connecting unit and the power unit while being galvanically isolated from said power unit and its connecting unit. In this embodiment, the terminals may be formed by terminal blocks to which the pairs of electrodes are connected, the active electronics of each acquisition system being embedded in the relay of the acquisition system. In this way, the cost of the measurement points is optimized. Provision may be made for each acquisition stage of a relay to be connected to one or more terminals.

Preferably, each second electrode of each acquisition system is connected to a common reference electric potential, in particular a ground of an acquisition stage of the relay of said acquisition system to which said second electrode is connected.

Provision may be made for each acquisition stage to comprise a multichannel analog-to-digital converter comprising a plurality of operational amplifiers and a plurality of elementary analog-to-digital converters, each operational amplifier being intended to receive the analog electric potentials acquired by a pair of electrodes of a group of electrodes and being arranged to measure the difference in potential between said analog electric potentials and each elementary analog-to-digital converter being arranged to convert one of said differences in potential into a digital signal. For example, each elementary analog-to-digital converter to be a delta-sigma analog-to-digital converter connected to the output of one of the operational amplifiers. A link between the output of an operational amplifier and a delta-sigma elementary analog-to-digital converter is understood to be both a single link between a single-output amplifier and the converter through which the signal amplified by the amplifier passes and also a dual link between a differential output amplifier and the converter where the amplified signal is obtained by the difference between the signals passing through each of the lines of the dual link.

For example, each operational amplifier may be a low-noise programmable gain amplifier (PGA). If applicable, each operational amplifier may be arranged to have a maximum gain of 24 and to introduce mid-level noise of less than 5 μV and in particular of substantially 0.1 μV in the case of an amplifier with a gain of 24. Advantageously, each delta-sigma elementary analog-to-digital converter is arranged to have a sampling rate of over 50 samples per second (SPS) or 50 Hz, or even of over 250 SPS or 250 Hz, and to convert an input signal received into a digital signal coded over 24 bits. If applicable, the overall unit formed by an operational amplifier and a delta-sigma elementary analog-to-digital converter is arranged to have an effective number of bits (ENOB) of at least 16, and in particular of at least 19 for an amplifier gain of 24 and for a sampling rate of 250 SPS. This combination is particularly suitable for the electrophysiological analysis of plants in that the differences in electric potential coming from the operational amplifiers are low-amplitude (approximately a microvolt) analog signals which may be amplified by the amplifier while introducing little noise and distortion owing to a low gain, and which however to be converted by the converter into high-resolution (approximately a nanovolt) digital signals.

Advantageously, each acquisition stage of the relay of each acquisition system is connected to a plurality of terminals of said acquisition system to receive said analog electric potentials collected by said terminals, said stage comprising a multiplexer arranged to multiplex said first analog electric potentials collected by said terminals. If applicable, the multiplexer of each acquisition stage is arranged to transmit the first multiplexed analog electric potentials to the multichannel analog-to-digital converter. The use of a multiplexer allows the number of terminals managed by the same relay to be increased, and therefore the number of measurement points to be reduced.

In another embodiment of the invention, each terminal of each acquisition system comprises at least an operational amplifier to measure said differences in potential between the analog electric potentials that it collects and to transmit said differences in potential to the relay of the acquisition system. If applicable, the relay of each acquisition system comprises a plurality of acquisition stages each connected to at least one of the terminals of the acquisition system to receive said differences in analog electric potentials, each acquisition stage being connected to the connecting unit and the power unit while being galvanically isolated from said power unit and its connecting unit. Provision may be made for the analog-to-digital converters to be arranged in the terminals or to be embedded in the relays. In this embodiment, although the cost of the measurement points increases, it can be ensured that the differences in electric potentials are measured and amplified as close as possible to the plants, such that the losses induced by the transmission by cable of said differences in electric potentials from the terminals to the relays are negligible and do not influence the data that is then transmitted to the central system. Similarly, it is thus possible to render negligible the noise that would be propagated on one of said cables, such as a leakage current or noise reflected from another cable.

Preferably, the connecting unit comprises a microcontroller arranged to serialize the digital signals converted by the analog-to-digital converters and transmitted by each of the acquisition stages to the connecting unit into a single digital signal and to transmit said single digital signal to the central system. Because of the microcontroller, it is possible to transmit the data coming from the analog-to-digital converters of each relay to the central system by the same cable. In a variant, said microcontroller could be replaced by an integrated circuit arranged to carry out said serialization, in particular by an FPGA.

In an embodiment of the invention, the central system is connected to each acquisition system by a data transmission network, the central system being arranged to transmit to each acquisition system signals containing electric power, in particular generated by the electric energy source, via the data transmission network. If applicable, the connecting unit of each acquisition system is arranged to transmit said digital signals to the central system via said data transmission network, and the power unit of each acquisition system is arranged to extract said electric power from the signals received from the central system via the data transmission network and to transmit said extracted electric power to the operational amplifiers and to the analog-to-digital converters. If applicable, the connecting unit and the power unit may form the same connecting unit to the data transmission network. Because of these characteristics, it is possible to pool the electric power supply and data transmission functions within the same cable connecting an acquisition system to the central system. Consequently, the number of cables needed to operate the analysis system can be reduced, which simplifies the installation and use of the system while increasing its robustness, in particular by reducing the number of connectors required.

Advantageously, the unit for connecting each acquisition system to the network comprises a connecter for connection to the central system by a cable of said data transmission network, for example an Ethernet cable, using power over Ethernet (PoE) functionality and in particular suitable for delivering electric power of at least 10 watts. This type of cable for this type of data transmission network comprises at least two wires, for example four pairs of twisted wires in the case of an RJ45 Ethernet cable, the data passing through at least one of the wires, in particular through at least two wires, the data being transmitted by the difference in potential of the two wires, and the electric power by at least two wires, in particular by at least two pairs of wires, one of the pairs providing a negative pole for the electric power and the other pair providing a positive pole for said electric power. In an example, the pair of wires transmitting the data may be one of the pairs of wires transmitting one of the poles for said electric power. In a variant, the pair of wires transmitting the data may be a different pair of wires from the pairs of wires transmitting the poles for said electric power.

In an embodiment of the invention, the connecting unit to the network of each acquisition system comprises a divider comprising at least two transformers arranged to emit and/or receive signals from the data transmission network, the divider being arranged to extract said electric power from said transformers. These may be isolation transformers, for example. If applicable, one of the transformers may be intended to be an uplink to transmit data from the central system to the unit for connecting to the network, the primary winding of said transformer being intended to be connected to one of the wires, for example to a twisted pair of wires, of the cable of the data transmission network. The other transformer may be intended to be a downlink to transmit the digital signal(s) generated by the acquisition system to the central system, the secondary winding of said transformer being intended to be connected to another of the wires, for example another pair of twisted wires, of the cable of the data transmission network. Advantageously, the unit for connecting to the network is arranged to extract electric power from the primary winding of the up transformer and from the secondary winding of the down transformer.

Advantageously, the unit for connecting to the network of each acquisition system comprises an analog-to-digital conversion and modulation/demodulation interface for the digital signal(s) generated by the acquisition system and the signals received from the central system. Said interface may be arranged between the divider and the analog-to-digital converters. Digital signal modulation should be understood as the transposition of said digital signal onto a carrier signal to transport information from said digital signal.

According to an embodiment of the invention, the connecting unit of each acquisition system comprises a coupler arranged to transmit the digital signals converted by the analog-to-digital converters of said acquisition system to the connecting unit, the coupler being arranged to galvanically isolate said analog-to-digital converters from the connecting unit. For example, the coupler may be an optocoupler.

Alternatively or cumulatively, each acquisition system comprises two rechargeable electric batteries and a switching system connected to said electric batteries, to the power unit, to the operational amplifiers and to the analog-to-digital converters of said acquisition system, the power unit of said acquisition system comprising a control unit of said switching system arranged to simultaneously:
a. connect one of the electric batteries to the operational amplifiers and to the analog-to-digital converters of said acquisition system and disconnect said electric battery from the power unit; and
b. disconnect the other electric battery from the operational amplifiers and from the analog-to-digital converters and connect said electric battery to the power unit.

For example, the power unit may comprise a power stage, in particular an electric power converter arranged to receive said electric power obtained from the electric energy source and to convert said power into electric power suitable for charging the electric battery.

In other words, for each acquisition system, the control unit allows the electric power supply of the active electronics of said acquisition system by one of the electric batteries and prevents the charging of said electric battery by means of said electric power obtained from the electric energy source and simultaneously prevents the electrical supply of the active electronics of said acquisition system by the other electric battery and allows the charging of said electric battery by means of said electric power obtained from the electric energy source. This characteristic allows the power unit in which disturbances of the electric power passing through the data transmission network are present to be isolated from the active electronics of the acquisition system, which may therefore determine the differences in potential and convert said differences in potential into digital signals without being disturbed.

If applicable, said reference electric potential is obtained by means of the electric battery supplying the acquisition device. The reference electric potential may in particular be obtained by means of the electric battery which is authorized by the switching system to supply the operational amplifiers and the analog-to-digital converters.

For example, the switching system comprises a first switching device connected to the power unit and to said electric batteries so as to connect the power unit selectively to one or other of the batteries and a second switching device connected to said electric batteries and to the operational amplifiers and the analog-to-digital converters so as to connect said operational amplifiers and analog-to-digital converters selectively to one or other of the batteries, the control unit being arranged to simultaneously control the state of the first and second switching devices so that said electric power obtained from the electric energy source is transmitted to one of the batteries and the operational amplifiers and analog-to-digital converters are supplied by the other battery. In other words, the first switching device allows one of the batteries to be recharged exclusively while the second switching device allows the operational amplifiers and analog-to-digital converters to be supplied exclusively by the other battery.

If applicable, the power unit may comprise one or more capacitors arranged to deliver electric power, when said capacitors are being discharged, to the operational amplifiers and analog-to-digital converters during changes of state of the switching devices.

In an embodiment of the invention, the electric energy source of the central system comprises a photovoltaic panel.

Advantageously, the central system comprises a voltage regulator connected to the photovoltaic panel. Said voltage regulator is arranged in particular to maintain the electric power and in particular the electric voltage supplied by the photovoltaic panel at a constant value. According to an embodiment, the voltage regulator may be a linear voltage regulator. If applicable, the voltage regulator may be a linear regulator with a low voltage dropout, also known as a low dropout (LDO) regulator. In another embodiment, the voltage regulator may be a DC/DC regulator, for example a voltage lowering regulator, in particular a Buck regulator.

In an embodiment of the invention, the central system comprises a rechargeable electric battery, the photovoltaic panel being arranged to charge said electric battery, which is therefore suitable for delivering said electric power to the acquisition systems. In a variant, the photovoltaic panel is arranged to deliver said electric power to the acquisition systems.

Advantageously, the photovoltaic panel is arranged to simultaneously charge the electric battery and deliver said electric power to the acquisition systems. If applicable, the system may comprise a unit for controlling the power supply of the acquisition systems, the control unit being arranged to selectively control the power supply of the acquisition systems by the photovoltaic panel and/or the electric battery, for example depending on whether the analysis system is operating diurnally or nocturnally, local sunshine information and/or information relating to the charge state of the electric battery.

Advantageously, the system may comprise a charge controller for said electric battery arranged to regulate the electric current generated by the photovoltaic panel.

Advantageously, the rechargeable electric battery has a nominal charge current value. In a first embodiment of the invention, the charge controller is arranged to control the value of the electric current generated by the photovoltaic panel so that said value corresponds substantially to said nominal charge current value. For example, the charge controller may be a chopper. In this embodiment, the aim is therefore to recharge the electric battery as quickly as possible. In a second embodiment of the invention, the charge controller is arranged to control the value of the electric current generated by the photovoltaic panel to a value substantially less than said nominal charge current value, for example less than 10%, in particular less than 5%, or for example, equal to 2.2% of said nominal charge current value. For example, the charge controller may be a current limitation diode. In this embodiment, as well as reducing the cost of the system, slow recharging of the electric battery is possible, which allows the noise from said recharging affecting the measurement of the electric signals emitted by the plant(s) by the first acquisition device to be reduced.

In an embodiment of the invention, the central system comprises a connecting module connected via the data transmission network to each acquisition system, the connecting module being suitable for receiving the electric power supplied directly or indirectly by the photovoltaic panel, in particular by means of the voltage regulator, and for transmitting the signals containing said electric power over said data network to each acquisition system. Preferably, the connecting module is suitable for receiving the digital signals transmitted by the connecting units of the acquisition systems over the data transmission network.

For example, the connecting module may be a PoE network switch, which may be incorporated in a computer server of the central system suitable for writing and reading data in said memory, the switch being connected to the photovoltaic panel, in particular by means of the voltage regulator. If applicable, each acquisition system may be connected by an Ethernet cable, or by a plurality of Ethernet cables connected to one another by one or more PoE repeaters, to a port of the network switch. In a variant, the network switch may be remote from said server and connected to said server by an Ethernet cable.

In a variant, the connecting module may comprise one or more PoE injectors connected on the one hand to a computer server of the central system suitable for writing and reading data in said memory and on the other hand to the photovoltaic panel, in particular by means of the voltage regulator. If applicable, each PoE injector may be connected to one of the acquisition systems by an Ethernet cable. In a variant, a PoE injector may be connected to a collector connected to a plurality of acquisition systems by a plurality of Ethernet cables.

Advantageously, the analysis system comprises at least one sensor, in particular a plurality of sensors, the or each sensor being suitable for measuring an environmental parameter of the field, and the or each sensor being connected to the central system. For example, the or each sensor may be chosen from the following types of sensor: an air or ground temperature sensor, an air or ground humidity sensor, a meteorological station, an atmospheric pressure sensor, an anemometer or a light meter. If applicable, the or each sensor may be connected to the central system by being connected to a relay of an acquisition system or directly to the central system.

Advantageously, the electrophysiological analysis system comprises a digital processing module for the digital signals generated by each acquisition system and transmitted to the central system to be stored there in memory.

Advantageously, the processing module may be arranged to implement one or more methods of processing the digital signal generated in order to determine a physiological state of the plant. It has been observed that the differences in potential thus measured and consequently the digital signals converted have variations that depend on the physiological state of the plant and in particular the sunshine received, water stress, heat stress, etc. These are variations in particular frequency components of said converted digital signals, in particular low amplitude variations of high-frequency components.

The processing module may be arranged to carry out spectral analysis of the digital signals. For example, the processing module may be arranged to obtain a spectrum from each digital signal (for example by means of a Fourier transform or by wavelet decomposition) and to determine one or more spectral power indicators from said spectrum, and in particular one or more indicators chosen from the following:
a. a spectral edge frequency (SEF), which is a frequency below which 95% of the total spectral power is concentrated;
b. a median edge frequency (MEF), which is a frequency below which 50% of the total spectral power is concentrated;
c. a power index, which is the total spectral power contained in a given frequency band, in particular a power index in the beta (>0.5 Hz), alpha (0.3-0.4 Hz), theta (0.1-0.2 Hz) and delta (0-0.1 Hz) frequency bands;
d. power index ratios, in particular a delta and a theta power index ratio;
e. power spectrum entropy (PSE).

Advantageously, the processing module may implement methods for analyzing variations in said indicator(s), in particular to recognize a shift in the spectral edge frequency and/or the median edge frequency according to a given model or alternatively a variation in a power index or a power index ratio according to a given model, or alternatively a variation in the power spectrum entropy according to a given model. It may, for example, be a method for classifying a variation in said indicator(s) from a library of predetermined variation models, each predetermined variation model being associated with a given physiological state of a plant. This type of classification method may be implemented for example by a pattern recognition algorithm based on automatic learning to recognize models in said model library.

The processing module may be remote from the central system or in a variant may be embedded in the central system, for example in a computer server of the central system.

The invention also relates to a system for electrophysiological analysis of a plurality of plants comprising a first acquisition device which comprises:
a. a plurality of first electrodes each intended to be connected to a different plant to pick up a first analog electric potential,
b. a first multiplexer of which each input is connected to one of the first electrodes, the first multiplexer being arranged to multiplex said first analog electric potentials picked up by the first electrodes into a first multiplexed analog signal, and
c. an analog-to-digital converter of which the input is connected to an output of the first multiplexer to receive said first multiplexed analog signal, the analog-to-digital converter being arranged to measure the difference in potential between said first multiplexed analog signal and a reference electric potential and to convert the measured difference in potential into a digital signal.

The invention also relates to a system for electrophysiological analysis of plants comprising:
a. a first acquisition device comprising at least a first electrode intended to be connected to a plant to pick up a first analog electric potential, the first acquisition device being arranged to generate a digital signal from said first analog electric potential;
b. a connecting unit arranged to emit and receive signals respectively to and from a data transmission network, the connecting unit being arranged to emit said digital signal to said data network and to extract electric power from the signals received from the data transmission network, said electric power being intended to supply the first acquisition device with electricity.

The invention also relates to a system for electrophysiological analysis of plants comprising at least a first acquisition device arranged to pick up electric signals emitted by one or more plants and to convert said electric signals into digital signals, characterized in that it comprises a photovoltaic panel arranged to supply said first acquisition device with electricity.

The present invention will now be described using examples that are purely illustrative and in no way limit the scope of the invention, and the accompanying drawings in which the different figures show:

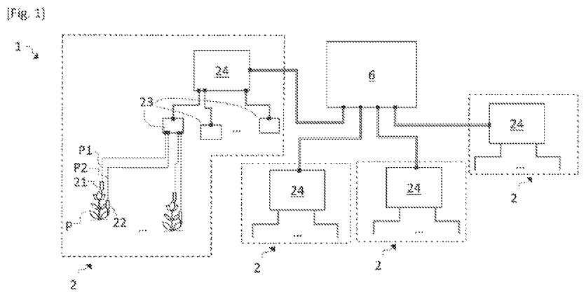

FIG. 1 shows diagrammatically and in part a system for electrophysiological analysis of plants according to an embodiment of the invention;

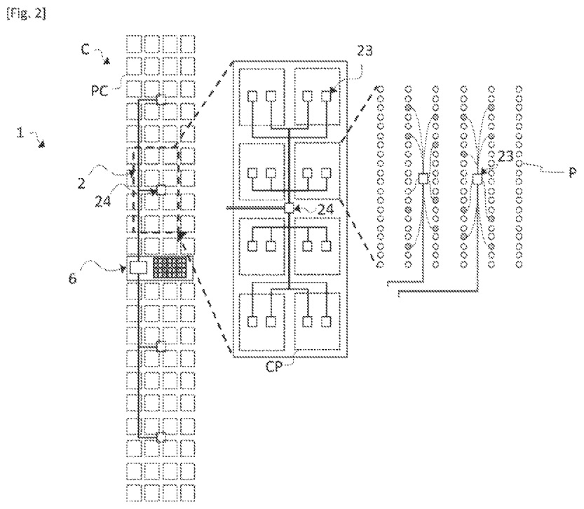

FIG. 2 shows diagrammatically and in part an example of deployment in a field of the system for electrophysiological analysis of plants in FIG. 1;

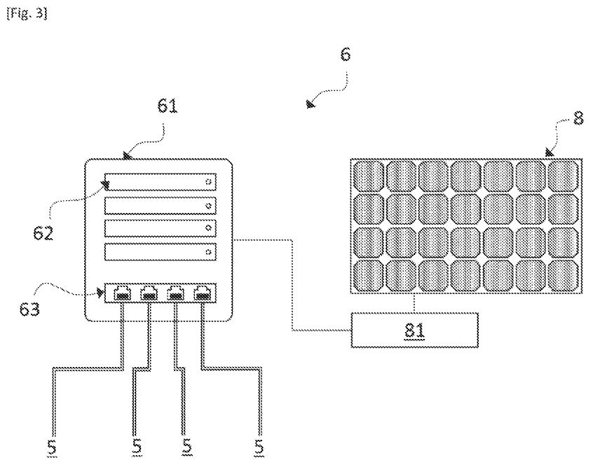

FIG. 3 shows diagrammatically and in part an embodiment of the central system of the system for electrophysiological analysis of plants in FIG. 1;

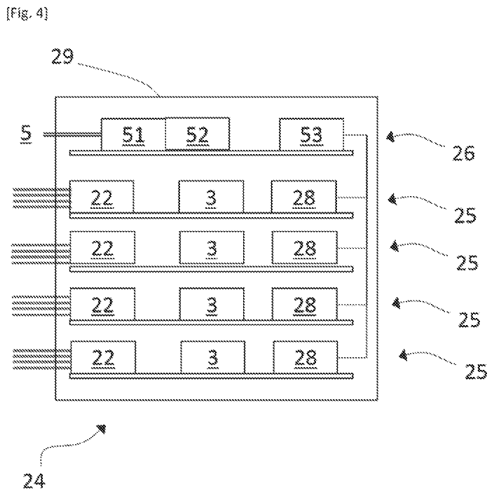

FIG. 4 shows diagrammatically and in part an embodiment of a relay of the system for electrophysiological analysis of plants in FIG. 1; and

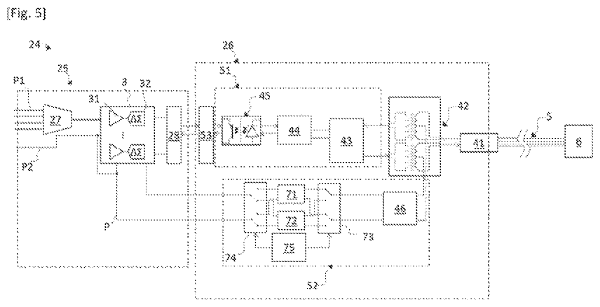

FIG. 5 shows diagrammatically and in part a diagrammatic view of the relay in FIG. 4.

In the description that follows, elements that are identical by structure or by function appearing in the different figures retain, unless stated otherwise, the same reference numerals.

FIG. 1 shows diagrammatically and in part a system for electrophysiological analysis 1 of a plurality of plants P according to an embodiment of the invention.

The system 1 is intended to be deployed in an agricultural field or in a glasshouse for measurements of analog electric signals emitted by the plants cultivated in said field or in said glasshouse to be carried out autonomously and continuously.

An example has therefore been shown in FIG. 2 of deployment of the system 1 in FIG. 1 in a field C made up of a plurality of parcels PC each comprising a plurality of plants P. As will be described, the system 1 allows the measurement of hundreds of said plants P spread over different parcels PC extending over a vast area of the field C to be carried out autonomously, in other words without the system 1 requiring an external energy source, and continuously for days or even weeks.

The system 1 comprises a plurality of acquisition systems 2 each connected to the same central system 6. In the example in FIG. 2, the system 1 comprises four acquisition systems 2, each the same as the others.

Each acquisition system 2 comprises a plurality of pairs of first electrodes 21 and of second electrodes 22, each pair being connected to one of the plants P. For example, the first electrode 21 is connected in the region of an upper portion of the stem of a plant P to be in contact with the phloem and the second electrode 22 is connected in the region of a lower portion of the stem of said plant P, also to be in contact with the phloem. In the example in FIG. 2, each acquisition system 2 comprises 128 pairs of electrodes 21 and 22.

Each first electrode 21 picks up a first analog electric potential P1 and each second electrode 22 picks up a second analog electric potential P2.

Each acquisition system 2 comprises a plurality of terminals 23 each connected to a group of electrode pairs 21 and 22. In the example in FIG. 2, each acquisition system 2 comprises sixteen terminals 23, each produced in the form of a terminal block to which eight pairs of electrodes 21 and 22 are connected, the terminal block thus collecting the first and second analog electric potentials P1 and P2 picked up by said eight electrodes.

Each acquisition system 2 comprises a relay 24 connected to the different terminals 23. As will be described below, each relay 24 comprises a plurality of operational amplifiers intended to measure the differences in potential between the first and second analog electric potentials P1 and P2 picked up by each pair of electrodes 21 and 22 of said acquisition system 2, as well as a plurality of analog-to-digital converters intended to convert said differences in potential into digital signals.

Each relay 24 further comprises a unit for connecting to a data transmission network to be connected to the central system 6 and to be able to transmit said digital signals to said central system 6. It will therefore be seen, with reference to FIG. 2, that the analysis system 1 is a network of sensors formed by the plants P, having a star topology around the central system 6, each acquisition system 1 having a tree topology of which the root is formed by the relay 24 and of which the endings are formed by the plants P. It will be seen that the number of relays 24 and terminals 23 described in FIG. 2 is an example, and a different, in particular a greater, number of relays and terminals may be envisaged without departing from the scope of the present invention. Similarly, adding concentrators between each relay 24 and the terminals 23 in order to increase the number of measurement points in the same zone of the field C, or alternatively adding extenders in order to reach parcels that are distant from the central system 6 may be envisaged.

As will be described below, the central system 6 comprises a computer server with an embedded memory in which are stored the different digital signals transmitted by the relays 24, which signals may then be processed in situ or alternatively reserved for subsequent processing. Moreover, to obtain an analysis system that can carry out measurements autonomously and continuously for a long period, the central system 6 comprises an electric energy source 8 that allows electric power to be supplied to the operational amplifiers and to the analog-to-digital converters of the relays 24. In the example in FIG. 2, the electric energy source 8 comprises a photovoltaic panel. Each relay 24 therefore comprises a power unit intended to receive said electric power transmitted by the central system 6 and to transmit said electric power to the operational amplifiers and to the analog-to-digital converter of said relay 24.

A preferred embodiment of the central system 6 of FIG. 1 and FIG. 2 will now be described with reference to FIG. 3.

As described previously, the central system 6 comprises a low-consumption, network-attached-storage (NAS) computer server 61, housing a plurality of hard disks 62 forming said memory of the central system 6. The system 1 also comprises a photovoltaic panel 8 associated with a linear low-dropout (LDO) voltage regulator 81 which regulates the electric power generated by the photovoltaic panel to supply electric voltage of a constant value to the server 61. Adding one or more electric batteries, connected on the one hand to the server 61 via a switching unit and on the other hand to the regulator 81 via a charge regulator, may be envisaged. In this case, a control unit may control the switching unit and the charge controller so as to allow the server 61 to be supplied directly in the diurnal period by the photovoltaic panel 8 and the electric battery or batteries to be charged, in which case supplying 61 the server by the electric batteries is not allowed, and so as to allow the server 61 to be supplied in the nocturnal period by the electric batteries.

The server 61 also has an embedded PoE network switch 63 to exchange data with the relays 24 of the acquisition systems 2 and in particular to receive the digital signals transmitted by said relays 24, and to be able to transmit the electric power generated by the photovoltaic panel 8 and the voltage regulator 81 to the operational amplifiers and to the analog-to-digital converters of said relays 24.

The relay 24 of each acquisition system 2 is thus connected to a port of said network switch 63 by an Ethernet cable 5, in other words a cable comprising for pairs of twisted wires, the data transmitted by the relays 24 or by the server 61 passing through two of the pairs and the electric power passing through the other two pairs.

A preferred embodiment of a relay 24 of an acquisition system 2 intended to be connected to the central system 6 of FIG. 3 will now be described with reference to FIG. 4 and FIG. 5.

The relay 24 comprises a plurality of acquisition stages 25 intended to receive the analog electric potentials P1 and P2 transmitted by the terminals 23, each comprising a plurality of operational amplifiers and analog-to-digital converters to measure the differences in potential and to convert said differences in potential into digital signals and a connecting stage 26 to which all said acquisition stages 25 are connected. Said connecting stage 26 comprises said connecting unit 51 which allows said digital signals to be transmitted to the central system 6 via the Ethernet cable 5 and said power unit 52 which allows the electric power transmitted by the central system 6 to be extracted from said Ethernet cable for distribution to the operational amplifiers and to the analog-to-digital converters of the acquisition stages 25. FIG. 4 therefore shows an overall view of a relay 24 while FIG. 5 describes diagrammatically one of the acquisition stages 25 and the connecting stage 26.

In the example in FIG. 4 and FIG. 5, each acquisition stage 25 is connected by a plurality of multiple coaxial cables to four terminals 23 to receive eight pairs of analog electric potentials P1 and P2 from each of said terminals. It will be observed that in the example described each second electrode 22 is connected to a common reference electric potential PR and in particular a ground of the acquisition stage 25 to which said second electrode 22 is connected, such that each second analog electric potential P2 is substantially equal to said common reference electric potential PR.

Each acquisition stage 25 comprises a multichannel multiplexer 27 of which the inputs are connected via the terminals 23 to the first electrodes 21. The multiplexer 27 is thus arranged to multiplex the first electric potentials P1 picked up by the first electrodes 21 and transmitted by the terminals 23. Each acquisition stage 25 therefore processes sequentially the first electric potentials P1 transmitted by the terminals 23, all the first electric potentials P1 transmitted by each terminal 23 being processed over the same multiplexing period.

Each acquisition stage 25 also comprises a multichannel analog-to-digital converter 3 comprising a plurality of operational amplifiers 31, each arranged to receive one of the outputs of the multiplexer 27, in this case a first analog electric potential P1 acquired by one of the first electrodes 21 and transmitted by one of the terminals 23, and said common reference electric potential PR and to measure the difference in electric potential between said first electric potential P1 and the common reference electric potential PR. In the example described, each operational amplifier 31 is a low-noise programmable gain amplifier (PGA), of which the gain may be programmed to a value of up to 24 while the average noise level introduced by the signal amplified by the amplifier does not exceed a value of substantially 2 μV for said gain of 24.

The multichannel analog-to-digital converter 3 also comprises a plurality of delta-sigma elementary analog-to-digital converters 32 each connected to the differential output of one of the operational amplifiers 31 to convert the difference in electric potential measured by said operational amplifier 31 into a digital signal. In the example described, each delta-sigma converter 34 is arranged to sample the difference in electric potential, for example at a sampling frequency of 250 Hz, so as to obtain a digital signal coded over 24 bits. This combination of low-noise PGA and delta-sigma converters allows an effective number of bits of at least 19 to be obtained when the PGA gain is fixed at 24. In this way, the digital signal obtained at the output of the elementary converter 32 has a particularly satisfying resolution, given the voltage level observed for the first electric potentials picked up by the electrodes 21. It should be noted that the sampling frequency may advantageously be modulated, in particular depending on the required recording period.

Finally, each acquisition stage 25 comprises a microcontroller 28 arranged to serialize the digital signals converted by each of the elementary converters 32, such that said acquisition system 25 is connected to the connecting stage 26 by a single channel over which said serialized digital signals pass. Similarly, the connecting stage 26 comprises a microcontroller 53 arranged to serialize the digital signals transmitted by all the acquisition stages 25. In the example described, all the components of each acquisition stage 25 are supported by the same printed circuit card, the acquisition stages 25 and the connecting stage therefore being stacked and housed in the same housing 29. All said elements therefore form a compact and autonomous assembly.

As can be seen, the connecting stage 26 of each relay 24 is arranged to galvanically isolate the acquisition stages 25 from the connecting and power units 51 and 52.

In the example described, the connecting stage 26 comprises an RJ45 connecter 41 to which the Ethernet cable 5 that connects said connecter to the central system 6 is connected.

To galvanically isolate the different components of the connecting stage 26 from other relays 24 and other sensors that may be connected to the central system 6 and to extract said electric power transmitted by the central system 6 via the Ethernet cable 5, the connecting stage 26 comprises a divider 42 comprising a plurality of isolation transformers, each connected via the connecter 41 to one of the twisted pairs in the region of the primary or secondary winding thereof. In FIG. 5, only the transformers connected to the twisted pairs transporting the electric power have been shown. The divider 42 is thus arranged to extract the electric power passing through the cable 5 from the windings of said transformers.

The connecting unit 51 comprises an interface 43 that allows the signals received from the cable 5 and the divider 42 to be converted into interpretable digital data and reciprocally the digital signals coming from the serializer 53 into signals that can be transmitted via the divider 42 and the connecter 41 over the cable 5.

The connecting unit 51 comprises a control unit 44 connected to the interface 43 arranged in particular to process the signals received from the cable 5.

The connecting unit 51 comprises an optocoupler arranged to transmit the digital signals coming from the serializer 53 to the control unit 44 while maintaining galvanic isolation between the control unit 44 and said serializer 53 and therefore the acquisition stages 25.

The power unit 52 comprises two rechargeable electric batteries 71 and 72 arranged, on the one hand, to supply the acquisition stages 25 with electricity and in particular the multichannel analog-to-digital converters 3, and on the other hand, to define the reference electric potential P.

The power unit 52 comprises an electric power converter 46 arranged to receive the electric power extracted from the divider 42 and convert said power into electric power suitable for charging the electric batteries 71 and 72.

The batteries 71 and 72 are therefore connected, on the one hand, to the power converter 46 and, on the other hand, to the acquisition stages by two switching devices 73 and 74 controlled simultaneously by a control unit 75 of the power unit 52.

The control unit 75 therefore controls the first switching device 73 so as to only allow the transmission of the electric supply power converted by the power converter 46 to one of the electric batteries, the battery 71 in the example in FIG. 5, in order to charge said battery. At the same time, the control unit 75 controls the second switching device 74 so as to only allow the transmission of the electric power contained in the other electric battery, the battery 72 in the example in FIG. 5 to the acquisition systems 25 for the electric supply thereof and the supply of the reference electric potential P2. This is because the inputs of the analog-to-digital converter 3 intended to receive the second analog electric potentials are connected via the second switching device 74 to the ground of said battery 72 supplying said acquisition stages 25.

In other words, the control unit 75 controls the switching devices 73 and 74 such that it is impossible for the battery 71 being charged to supply the acquisition stages 25 with electricity and such that it is impossible for the battery 72 supplying acquisition stages 25 and supplying the reference potential P2 to be charged by the power converter 46.

It can therefore be seen from FIG. 5 that, because of the switching devices 73 and 74 and the optocoupler 45, the reference potential P2 allowing the digital signals to be generated is independent of any disturbance that could occur on the Ethernet cable 5 and in particular any disturbance of the ground of the electric supply passing through said cable.

It should be noted that the analysis system 1 may comprise a digital processing module for the digital signals stored in the memory of the server 61, which is arranged to implement one or more methods of processing said digital signals in order to determine the physiological state of the plants P. Although not limiting, the processing module may be arranged to obtain a spectrum from each digital signal (for example by means of a Fourier transform or by wavelet decomposition) and to determine one or more spectral power indicators from said spectrums, then to implement methods for analyzing variations in said indicator(s) in order to determine the physiological state of the plants P. Said digital processing module may be embedded in the server 61 or may be remote from the server 61.

The description above explains clearly how the invention allows the objects set to be achieved, in particular by proposing a system for electrophysiological analysis of plants comprising a plurality of measurement points organized in a star architecture around a central system.

In any event, the invention is not limited to the embodiments specifically described in this document, and extends in particular to all equivalent means and to any technically possible combination of said means. Provision may be made in particular for the operational amplifiers and/or the analog-to-digital converters to be arranged in the region of the terminals of the acquisition systems, so as to render negligible the losses induced by the length of the entirety of the cables connecting the electrodes to the central system. Other embodiments may also be envisaged that allow the electric power from the photovoltaic panel to be transmitted to the relays of the acquisition systems, in particular by using concentrators and/or PoE injectors remote from the server of the central system. Replacing one or more of the terminals and/or relays by a sensor suitable for measuring an environmental parameter of the camp may also be envisaged. Not connecting the second electrodes to the same common reference electric potential and adding a second multiplexer to the acquisition stages could also be envisaged to allow the second analog electric potentials picked up by said second electrodes to be multiplexed.

1. A system for electrophysiological analysis of plants, comprising:
a. a plurality of acquisition systems, each acquisition system comprising:
i. a plurality of pairs of electrodes each comprising a first electrode and a second electrode each intended to be connected to the same plant in order to pick up first and second analog electric potentials;
ii. a plurality of operational amplifiers arranged in order to measure the differences in potential between the first and second analog electric potentials acquired by the first and second electrodes of said acquisition system and a plurality of analog-to-digital converters designed to convert the differences in potential into digital signals;
b. a central data collection system comprising a memory and a source of electrical power;
and in that each acquisition system is connected to the central system and comprises:
c. a connecting unit designed to transmit the digital signals to the central system;
d. a power unit designed to transmit electrical power obtained from the electrical energy source to the operational amplifiers and to the analog-to-digital converters;
and in which each acquisition system is designed to galvanically isolate the operational amplifiers and the analog-to-digital converters from the power unit and the connecting unit.

2. The system according to claim 1, wherein each acquisition system comprises a plurality of terminals each connected to a group of electrode pairs of said acquisition system and arranged to collect said analog electric potentials picked up by the first and second electrodes of said group, and a relay connected to said terminals of said acquisition system and to the central system, said relay comprising said connecting unit and said power unit, wherein the operational amplifiers and the analog-to-digital converters of each acquisition system are provided in the relay and/or in the terminals of said acquisition system, wherein the relay of each acquisition system is arranged to galvanically isolate said operational amplifiers and said analog-to-digital converters of said acquisition system from its power unit and from its connecting unit.

3. The system according to claim 1, wherein each terminal of each acquisition system is arranged to transmit the analog electric potentials that it collects to the relay of said acquisition system, wherein the relay of each acquisition system comprises a plurality of acquisition stages each connected to at least one of the terminals of said acquisition system to receive said collected analog electric potentials and comprising at least an operational amplifier and an analog-to-digital converter to measure said differences in potential and convert said differences in potential into digital signals, each acquisition stage being connected to the connecting unit and the power unit while being galvanically isolated from said power unit and its connecting unit.

4. The system according to claim 1, wherein each acquisition stage of the relay of each acquisition system is connected to a plurality of terminals of said acquisition system to receive said analog electric potentials collected by said terminals, said stage comprising a multiplexer arranged to multiplex said first analog electric potentials collected by said terminals.

5. The system according to claim 2, wherein each terminal of each acquisition system comprises at least an operational amplifier to measure said differences in potential between the analog electric potentials that it collects and to transmit said differences in potential to the relay of said acquisition system, and wherein the relay of each acquisition system comprises a plurality of acquisition stages each connected to at least one of the terminals of said acquisition system to receive said differences in analog electric potentials, each acquisition stage being connected to the connecting unit and the power unit while being galvanically isolated from said power unit and its connecting unit.

6. The system according to claim 2, wherein the connecting unit comprises a microcontroller arranged to serialize the digital signals converted by the analog-to-digital converters and transmitted by each of the acquisition stages to the connecting unit into a single digital signal and to transmit said single digital signal to the central system.

7. The system according to any claim 1, wherein the central system is connected to each acquisition system by a data transmission network, wherein the central system is arranged to transmit to each acquisition system signals containing electric power via the data transmission network, and wherein the connecting unit is arranged to transmit said digital signals to the central system via said data transmission network, and wherein the power unit is arranged to extract said electric power from the signals received from the central system via the data transmission network and to transmit said extracted electric power to the operational amplifiers and to the analog-to-digital converters.

8. The system according to claim 1, wherein the connecting unit of each acquisition system comprises a coupler arranged to transmit the digital signals converted by the analog-to-digital converters of said acquisition system to the connecting unit, the coupler being arranged to galvanically isolate said analog-to-digital converters from the connecting unit.

9. The system according to claim 1, wherein each acquisition system comprises two rechargeable electric batteries and a switching system connected to said electric batteries, to the power unit, to the operational amplifiers and to the analog-to-digital converters of said acquisition system, the power unit of said acquisition system comprising a control unit of said switching system arranged to simultaneously:
connect one of the electric batteries to the operational amplifiers and to the analog-to-digital converters of said acquisition system and disconnect said electric battery from the power unit; and
disconnect the other electric battery from the operational amplifiers and from the analog-to-digital converters and connect said electric battery to the power unit.

10. The system according to claim 1, wherein the electric energy source of the central system comprises a photovoltaic panel.
